(19) United States
(12) Patent Application Publication
(10) Pub. No.: US 2026/0345092 A1
(43) Pub. Date: Nov. 5, 2026

(54) CONTINUOUS CUFFLESS BIOPARAMETER MONITORING

(71) Applicant: Worcester Polytechnic Institute, Worcester, MA (US)

(72) Inventors: Edward A. Clancy, Worcester, MA (US); Rajesh S. Kasbekar, Worcester, MA (US)

(21) Appl. No.: 19/548,305

(22) Filed: Feb. 24, 2026

Related U.S. Application Data
(60) Provisional application No. 63/762,236, filed on Feb. 24, 2025.

(51) Int. Cl. A61B 5/00; A61B 5/021; A61B 5/1455

(52) U.S. Cl. CPC A61B 5/7278; A61B 5/021; A61B 5/14551; A61B 5/7267; A61B 5/7275; A61B 2560/0223

(57) ABSTRACT

A cuffless blood pressure measurement approach computes a blood pressure from a related physiological parameter such as a photoplethysmographic (PPG) measurement provides a less invasive measurement for determination of blood pressure (BP). Nocturnal readings of blood pressure are achievable without waking the patient, as PPG readings are obtainable from a finger probe, rather than a conventional invasive blood pressure measurement. A model of features related to blood pressure trained with blood pressure measurements computes the blood pressure (systolic and diastolic values) based on the features. The features include a personalization derived from an actual blood pressure baseline. Computational “drift” or deviation from accurate BP is detected from a deviation in the PPG waveform, or measured values, indicating a need for a refreshed actual (cuff) BP measurement for refreshing the baseline. However, baseline refresh readings occur infrequently for allowing a substantial duration of uninterrupted intervals between cuff readings.

### RELATED APPLICATIONS

This patent application claims the benefit under 35 U.S.C. § 119 (e) of U.S. Provisional Patent App. No. 63/762,236, filed Feb. 24, 2025, entitled “CONTINUOUS CUFFLESS BIOPARAMETER MONITORING,” incorporated herein by reference in entirety.

### BACKGROUND

Blood pressure monitoring has traditionally taken the form of a pressurized cuff and pressure gauge in conjunction with a stethoscope and manual monitoring of the cuff pressure by a medical technician (e,g, doctor or nurse). Modern enhancements have automated the pressurization and monitoring, but still rely on the inflatable cuff to surround the patient arm, and requires iterative cycles to gather blood pressure (BP) statistics over time. Nocturnal measurement of a sleeping patient is impractical due to repeated cuff inflation interfering with sleep patterns.

### SUMMARY

A cuffless blood pressure measurement approach computes a blood pressure from a related physiological parameter such as a photoplethysmographic (PPG) measurement of blood oxygen and provides a less invasive measurement for determination of blood pressure (BP). Nocturnal readings of blood pressure are achievable without waking the patient, as PPG readings are obtainable from an optical based finger probe, rather than a conventional compressed cuff around the upper arm. A model of features related to blood pressure trained with blood pressure measurements computes or predicts the blood pressure (systolic and diastolic values) based on the features. The features include a personalization derived from an actual blood pressure baseline. Computational “drift” or deviation from accurate BP is detected from a deviation in the PPG waveform, or measured values, indicating a need for a refreshed actual (cuff) BP measurement for refreshing the baseline. However, baseline refresh readings occur infrequently for allowing a substantial duration of uninterrupted intervals between cuff readings.

Configurations herein are based, in part, on the observation that blood pressure is a commonly monitored parameter, particularly in hospital or inpatient settings. Continual BP readings are often employed for critical or unstable patients, using an invasive strain gage or such other blood pressure sensor. Variations in the cuff pressure are observed or measured by manual or electronic means to determine systolic and diastolic blood pressure.

When patient status so requires, intermittent blood pressure readings are continued at regular intervals via cuff inflation, causing an invasive interruption of sleep cycles. Conventional approaches have sought to obtain BP readings indirectly via other parameters, such as photoplethysmography readings. Unfortunately, conventional approaches to correlate PPG readings with BP suffer from the shortcoming that the PPG-computed BP readings are subject to “drift,” a slight inaccuracy over time, and requiring a repeated, true, cuff measurement BP readings, still at regular intervals. Accordingly, configurations herein substantially overcome the shortcomings of conventional approaches by augmenting and refining the PPG readings with additional features such as values derived from the true cuff reading or any other direct blood pressure measurement method such as an invasive strain gage measurement or tonography measurement to personalize the computed BP. Such personalization features are based on the actual BP to improve the computed BP such that the PPG computed BP remains valid for an extended period, preferably an entire overnight sleep cycle, between cuff based or other such direct BP refresh measurements of actual BP. An evaluation of a waveform of the PPG measurement is evaluated to determine when the PPG reading varies significantly enough to compromise the computed BP and trigger a direct BP measurement to reset a baseline of BP.

In further detail, a method of measuring physiological parameters includes receiving a stream of values indicative of a physiological parameter, and comparing the stream of values to a database or model of features related to the physiological parameter. The model is used to predict, based on the comparison, a series of values of the physiological parameter, and determines when the predicted series of values is no longer an accurate reflection of the physiological parameter for commencing a refreshed baseline reading.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is an example clinical environment for BP monitoring suitable for use with configurations herein;

FIGS. 2A and 2B show respective training and deployment usage scenarios in the environment of FIG. 1;

FIG. 3 shows a flowchart of model training as in FIG. 2A;

FIGS. 4A and 4B show results of computed BP compared to actual measurements using the model as in FIGS. 2A and 3; and

FIG. 5 shows a flowchart of model deployment as in FIG. 2B.

### DETAILED DESCRIPTION

Depicted below are example configurations of a cuffless blood pressure measurement system suitable for use with the disclosed approach for cuffless blood pressure monitoring using non-invasive photoplethysmography measurements and a BP model to compute systolic and diastolic BP. The BP model (model) is trained on features related to or indicative of BP, and computes, predicts, or classifies the BP values based on the measured physiological parameter such as PPG blood O₂ and the model.

Nocturnal monitoring of continuous, cuffless blood pressure (BP) can provide a beneficial improvement for prognostication of cardiovascular and other diseases, due to its strong predictive capability. Nevertheless, the lack of an accurate and reliable method, primarily due to confounding variables, has prevented its widespread clinical adoption. One approach to correlate physiological features other than BP cuff readings include the so-called “Catch-22” set of features (Lubba, C., H., Sethi, S. S., Knaute, P., Schultz, S. R., Fulcher, B, D., Jones, N., “Catch22: Canonical Time-series Characteristics,” 2019). Configurations herein demonstrate an additional set of personalization features to prolong an interval over which the PPG signal can be leveraged for providing accurate BP readings. Personalization is a calibration that finetunes the model using a portion of the target patient's data including the mean and standard deviation (SD) of the PPG signal used for BP computation.

Configurations herein demonstrate how optimized machine learning using the so-called “Catch-22” features, when applied to a photoplethysmogram (PPG) waveform and personalized with direct BP data through transfer learning, can accurately estimate systolic and diastolic BP.

In a particular example configuration, following training with a hemodynamically compromised “calibration-free” dataset (n=1293), the systolic and diastolic BP tested on a distinct dataset that met AAMI criteria (n=116) had acceptable error biases of −1.85 mm Hg and 0.11 mm Hg, respectively [within the 5 mm Hg IEC/ANSI/AAMI 80601-2-30, 2018 standard], but standard deviation (SD) errors of 19.55 mm Hg and 11.55 mm Hg, respectively [exceeding the stipulated 8 mm Hg limit]. In an example configuration, the employed dataset is “VitalDB,” (Lee, H.-C.; Park, Y.; Yoon, S. B.; Yang, S. M.; Park, D.; Jung, C.-W. “VitalDB, a high-fidelity multi-parameter vital signs database in surgical patients”. Sci. Data 2022, 9, 1-9.

However, the personalization as disclosed herein using an initial calibration data segment and subsequent use of transfer learning to fine-tune the pretrained model produced acceptable mean (−1.31 mm Hg and 0.10 mm Hg) and SD (7.91 mm Hg and 4.59 mm Hg) errors for systolic and diastolic BP, respectively. Use of a Levene's test for variance found that the personalization method significantly outperformed (p<0.05) the calibration-free method, but there was no difference between three evaluated machine learning methods. Optimized multimodal Catch-22 features, coupled with personalization, demonstrate great promise in the clinical adoption of continuous, cuffless blood pressure estimation in applications such as nocturnal BP monitoring.

Cuffless blood pressure estimation based on continuous measurements, e.g., using the PPG and electrocardiogram (ECG) signals rather than the intermittent cuff-based measurement of BP, has gathered significant attention recently. PPG is an optical measurement of tissue underneath the sensor and reflects heart activity non-intrusively and non-invasively. While the intent of such BP monitoring is to minimize patient discomfort and harness the significant advantages of continuous measurement compared with intermittent clinical cuff BP measurements taken during the day, there is a more compelling reason to remove any barriers related to its clinical adoption: nighttime or nocturnal BP.

Continuous measurement of nocturnal BP is a far better predictor of cardiovascular disease and stroke than daytime BP. Furthermore, nocturnal hypertension can occur in people whose daytime BP is normal. Spikes in BP during sleep can have potentially serious health implications that include heart failure and other such cardiovascular or renal diseases. Nighttime BP, therefore, has strong prognostic value in predicting outcomes. It allows for the diagnosis of masked hypertension due to isolated nocturnal hypertension. It offers information on cardiovascular modulation during sleep for both healthy and diseased conditions. In addition, monitoring BP during the night can help measure the effect of drug- or device-based treatment over a 24 h period. Nonetheless, nocturnal hypertension is not easy to measure as routine BP checks are mostly taken during daytime hours. Because it is not practical to make frequent cuff-based BP measurements at night, an accurate and reliable method for cuffless and continuous BP measurement is critical for the measurement of nocturnal BP.

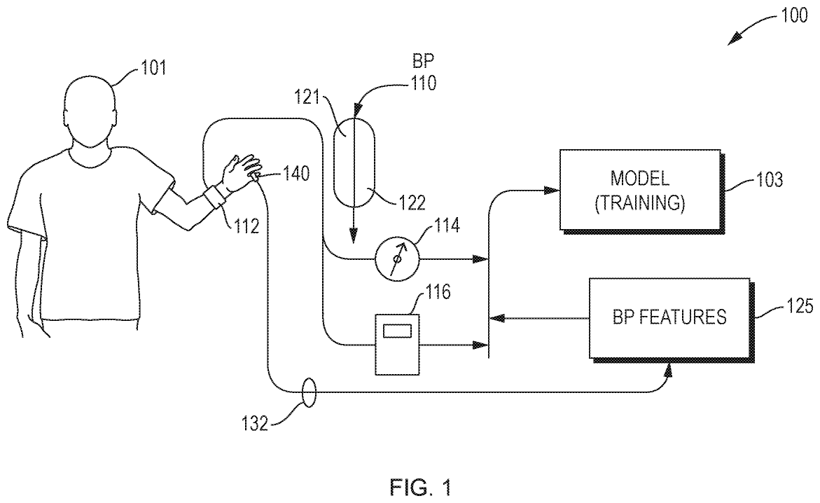

FIG. 1 is an example clinical environment 100 for BP monitoring suitable for use with configurations herein. Referring to FIG. 1, a patient or subject 101 receives an initial or baseline BP measurement (reading) 110 from a pressurized cuff 112 around the arm and an analog gauge 114 or electronic sensing 116 (e.g. transducer). The BP values 110, typically defined as systolic 121 (SBP) and diastolic 122 (DBP) values, combine with other BP features 125 for training a model 130. The model 130, once trained, can predict or compute BP values for blood oxygenation obtained from pulse oximetry or other suitable physiological parameter.

For training the model 130 for predicting BP from blood oxygenation, or PPG readings, values in a stream 132 of values are based on a measurement of dissolved blood gases from a fingertip sensor projecting light through a digit to detect a percentage of oxygenated (oxygen bound) hemoglobin, according to well established PPG approaches. The stream of values 132 therefore includes pulse oximetry waveform readings received from a subject, and the predicted series of values define a blood pressure of the subject.

In the model 130, contained physiological parameters further include the systolic blood pressure, diastolic blood pressure, and personalization parameters including a baseline value derived from a previous systolic and diastolic blood pressure. In the example configuration, the model 130 therefore includes blood pressure parameters configured for predicting a systolic blood pressure and a diastolic blood pressure, where the model 130 is trained on a dataset of features including a mean of a photoplethysmography measurement of blood oxygen and a standard deviation of a photoplethysmography measurement of blood oxygen, for subsequent use in PPG computation of cuffless BP values.

Measurement of cuffless, continuous BP using PPG has encountered challenges in conventional approaches. Clinical adoption of cuffless devices has been slow due to the issue of trusting the measurement in the individual patient. To address this need, the European Society of Hypertension created revised recommendations for the validation of cuffless BP measuring devices, with the goal of providing a standard for clinical acceptance of such devices. These recommendations include six stepwise device-specific tests depending upon device type and include both static and dynamic tests as well as tests based on device position, exercise, treatment, sleep, and re-calibration. They are based on a comprehensive, demanding, and complex methodology built on established principles from the American Association of Medical Instrumentation (AAMI), The International Organization for Standardization (ISO), and European Society of Hypertension.

Conventional approaches demonstrate that accurate and reliable estimation of BP in a cuffless device that is reliable for long time periods is challenging, with SBP proving more difficult to estimate than DBP. Cuffless BP estimation may be attainable using demographic information and features extracted from (i) pulse arrival time (PAT) or pulse transit time (PTT) and (ii) pulse wave morphology (PWM). The use of a high-quality PPG signal selective features, and an optimal machine learning algorithm can provide promising results in estimating SBP and DBP. One motivation for the present configuration is to provide a reliable long-term, clinically acceptable method using a three-fold strategy: (i) to evaluate performance improvements when using over 1500 subjects from the VitalDB (PulseDB) database; (ii) to investigate a state-of-the-art feature extraction algorithm called Catch-22 (CAnonical Time-series Characteristics), selected through highly comparative time-series analysis on PPG waveforms; and (iii) to measure the impact of targeted personalization by using the SBP and DBP obtained from the test subject as personalization and feed it back into the general training algorithm through trans-fer learning. Catch-22 is a method of extracting a relatively small set of relevant features from the pantheon of features utilized in time-series analysis. This small set of features (including linear and non-linear autocorrelation, successive differences, value distributions and outliers, and fluctuation scaling properties) has exhibited strong classification or regression performance in past studies and is minimally redundant. Personalization is a calibration that fine-tunes the general models using a small segment of the target subject's data consisting of the mean and SD of the BP signal. The data are used for calibration in the training dataset and for insertion into the main algorithm layers using transfer learning. Alternatively, a calibration-free method also exists that does not use any target subject data for training or insertion into the main algorithmic layers. One hypothesis is that the use of this large dataset, along with the selective features on key waveform inputs and personalization, significantly enhances the accuracy for cuffless SBP and DBP, comparing with intra-arterial BP as the gold standard and ensuring its reliability for continuous applications.

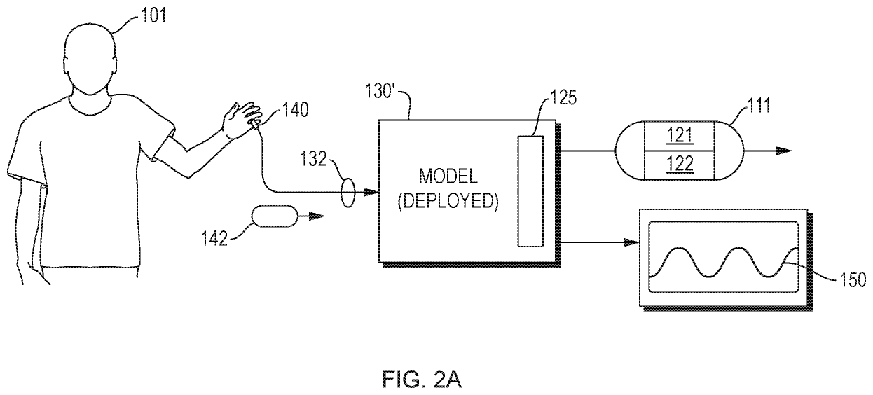

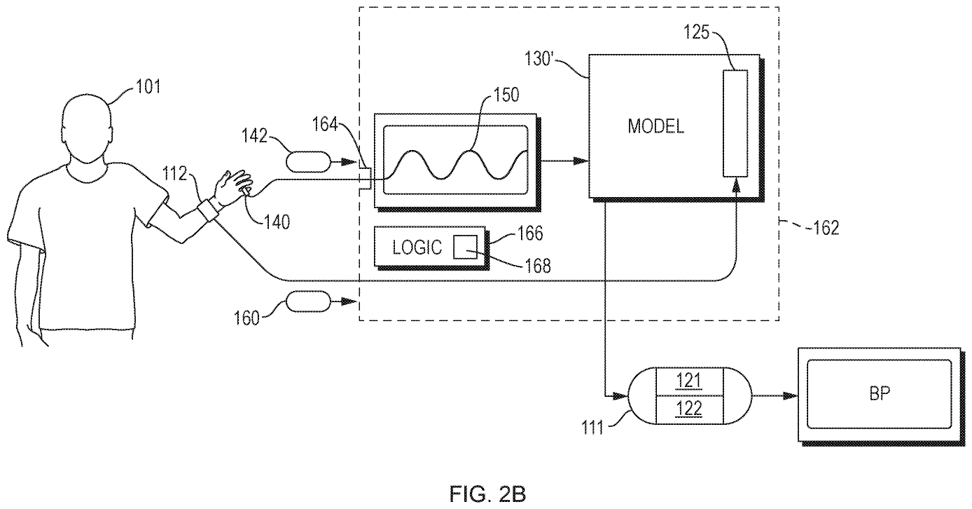

FIGS. 2A and 2B show respective training and deployment usage scenarios in the environment of FIG. 1. FIG. 2A depicts the 130′ trained using PPG features and personalization features indicative of patient specific BP values and information. The personalization features used for training the model include a mean of a photoplethysmography measurement of blood oxygen and a standard deviation of a photoplethysmography measurement of blood oxygen corresponding to at least one of a systolic blood pressure and the diastolic blood pressure. Referring to FIGS. 1 and 2A, a blood oxygenation sensor 140 senses PPG values 142 indicative of blood pressure. As the human heart pumping mechanism operates on a series of muscular contraction “pulses,” or beats, each beat defines a systolic peak of highest pressure (the upper number in a typical BP reading). The valley between these peaks defines the low pressure diastolic value (bottom number). As PPG readings detect hemoglobin-bound oxygen passing a point in bloodflow, a variance between the systolic and diastolic peaks occurs. These PPG values, along with other personalization features 125 based on actual SBP and DBP, allow the PPG readings alone to accurately predict BP in the trained model. The model 130′ receives the sensed PPG values 142, and computes or predicts the BP values 111. The PPG values therefore define a PPG waveform 150 that can be used to compute the BP 111.

The model 130 is therefore trained not only on the Catch-22 features discussed above, but also with the personalization features 125. The personalization features 125 allow increased longevity of accurate BP readings 111 based on PPG values 142 before an actual cuff BP or any other direct BP measurement 110 is invoked to recalibrate. In a particular configuration, generation of the trained model 130′ was developed as follows.

In one configuration, a data corpus by Wang (Wang, W.; Mohseni, P.; Kilgore, K. L.; Najafizadeh, L. PulseDB: “A large, cleaned dataset based on MIMIC-III and VitalDB for benchmarking cuff-less blood pressure estimation methods.” Front. Digit. Health 2023, 4, 1090854) pre-processed and extracted data from the larger PulseDB database to create the VitalDB database. The PulseDB database is the largest filtered and cleaned database to date that enables a standardized, reliable, and reproducible evaluation of cuffless BP estimation models. VitalDB contains hemodynamically compromised patient recordings (with demographic information) of continuous waveform ECG (Electrocardiogram), PPG, and ABP (Arterial Blood Pressure) data, sampled at 125 Hz, recorded over 5 to 10 days from ICU patients who underwent surgeries in the Seoul National University Hospital, South Korea. The criteria used by Wang et al. for extraction included the presence of ECG lead-II, fingertip PPG, and ABP signals. Wang et al. extracted, filtered, and cleaned data for invalid samples, saturated and flatline signals, and quality of the signals. The R-wave peaks of the ECG signals were detected for each record using the Pan-Tompkins QRS detection algorithm. Systolic peaks of the PPG signal were located using Elgendi's algorithm (a time-domain method designed for fast and accurate R-peak detection in electrocardiogram (ECG) signals and systolic peak detection in photoplethysmogram (PPG) signals), and diastolic valleys were located as the minimum between every two consecutive systolic peaks. Next, Wang et al. divided each signal (ECG, PPG, and ABP) into contiguous 10 s segments that were, on average, 9 min apart. Segments having more than three consecutive samples of the same value equaling the minimum or maximum amplitude within the segment, or more than 1 s of the same amplitude (i.e., saturated/flatlined) for any signal, were removed. Reference SBP and DBP values of each segment were defined as the average beat-to-beat SBP and DBP values within that segment. Configurations herein further removed the ECG, PPG, and ABP signals with less than 3 peaks.

A subset of 1525 subjects was assembled that met the American Association of Medical Instrumentation (AAMI) test data inclusion criteria to ensure a uniform representation of hypotensive and hypertensive subjects. The 1525 subjects were sub-divided by Wang into 1293 subjects for training, 116 for validation, and 116 for testing. The AAMI criteria were applied to the test dataset and mandate at least 85 subjects in this test dataset. At least 5% of these subjects must belong to each of the following categories: an SBP below 100 mm Hg, an SBP above 160 mm Hg, a DBP below 60 mm Hg, and a DBP above 100 mm Hg. In addition, at least 20% of the subjects must each have an SBP over 140 mm Hg and a DBP over 85 mm Hg. The test dataset met these criteria. Per subject, we randomly selected 19, 7, and 7 “10-s segments” for each of training, validation, and testing, respectively. The 19 segments were randomly chosen because for 1293 subjects, they gave a total of 24,567 data segments, which were well above fit parameter requirements (10 times 840 fit parameters or 8400) of the deep learning ResNET algorithm. As detailed below, only 3 test values per subject were evaluated for estimation error from the 7 available test segments as stipulated by the AAMI criteria. Seven segments were randomly chosen and arranged in numeric order because the first segment was used for personalization, 3 test values per subject were required by the AAMI criteria, and 3 additional data segments had to be extracted to maintain independence of the direct pressure calibration values if the flow criteria were not met in the initial 3 segments, as explained below.

From every 10 s data segment, a “Catch-22” set of the 22 features was extracted. This set includes a generation of small, canonical subsets of features that display high performance across a given ensemble of tasks. They also exhibit complementary performance characteristics with each other. Catch-22 uses linear and non-linear autocorrelation, successive differences, value distributions, outliers, and fluctuation scaling to extract features that reduce dimensionality, are minimally redundant, and facilitate feature-based time-series analysis.

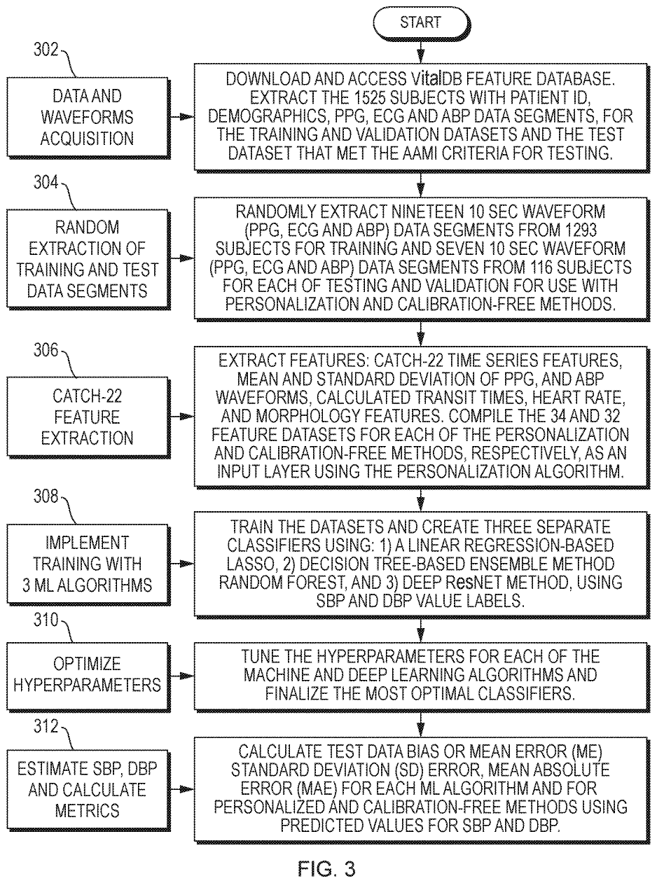

In addition to the Catch-22 features, we also extracted the following features: the mean and SD of the PPG, two temporal features (Pulse Arrival Time (PAT) and heart rate), and four PPG morphology features. The PAT was extracted from each data record by computing the average of the first 3-time intervals between the ECG R-peak to the ensuing PPG valley. The heart rate was derived from the average time interval for the same three consecutive ECG R-peak to R-peak intervals. The four PPG morphology features were extracted as an average from three successive beats, randomly selected. The features were the average time interval over 3 beats between the valley and peak of the PPG waveform, the average time interval over 3 beats between the peak of the same PPG waveform and valley of the subsequent PPG waveform, the peak amplitude over 3 beats, and the derivative of the PPG peak amplitude computed as the average of the slope of the line connecting the valley to the peak over 3 beats. We also used the demographic features of age and sex, giving a total of 32 input features for the calibration-free method. For the personalized dataset only, we also utilized in a unique manner the two additional features of ABP mean and standard deviation, as the VitalDB data did not contain any cuff BP measurements. In practice, a systolic and diastolic reading obtained from a cuff, or any other reliable direct BP measurement method, would be used to substitute for these ABP features. This resulted in the use of a total of 34 input features for personalization. These same set of features were analyzed for relevance using Shapely analysis and then used in all three ML algorithms to enable a comparison of the performance among the three algorithms. FIG. 3 below shows a schematic diagram of the flow steps consisting of feature extraction, training, hyperparameter selection, and testing.

FIG. 2B shows an example of a clinical application of the trained model 130′ for using the personalization features 125 in conjunction with a stream of PPG values for computing BP values 111, and deferring the baseline or calibration “refresh” so as to not wake or disturb a sleeping patient.

Referring to FIGS. 1-2B, a method of measuring physiological parameters, such as BP 111, includes, receiving the stream of values 142 indicative of a physiological parameter, and comparing the stream of values 142 to a database, defined by trained model 130′, of features related to the physiological parameter. It should be apparent that the stream of values 142, such as PPG measurements, is a stream of readings generated at regular intervals, as are the stream of predicted BP values 111. The stream of values 142 therefore defines the PPG waveform 150. This waveform exhibits statistical parameters, such as mean, standard deviation, gain and offset. The statistical parameters that can be used to identify the drift in accuracy and the need to “reset” or calibrate with a cuff BP read or an alternate direct BP measurement method. The model 130′ predicts, based on the comparison with the stream of PPG values 142, a series of values of the physiological parameter such as BP, as a repeated “cuffless” BP measurement 111 over time. The approach encompasses a full system 162 that determines when the predicted series of values 111 is no longer an accurate reflection of the physiological parameter, and commences a cuff-based BP measurement 160 for providing a refreshed BP measurement 160. The system 162 may take the form of a device having an interface 164 to the O₂ sensor 140, a processor 168 and logic 166 for receiving and comparing the PPG values 142.

The full system 162 may comprise a suitable computing platform for receiving both the PPG values 142 and the cuff measured BP or other direct BP measurement values 110 from a refresh reading 160. In response to determining an inaccuracy of the physiological parameter, the system 162 recalibrates the stream of values by performing a true measurement of the physiological parameter with a true BP measurement 110. Thus, the full incoming stream of values to the model 130′ includes a personalization value derived from previous actual measurements of the physiological parameter (such as BP). Typically monitoring commences with a baseline value derived from an actual measurement from a patient to which the physiological parameter applies, and which is refreshed from a true measurement of the physiological parameter (BP 110).

It follows that the frequency of a refresh/baseline reading 160 or cuff/direct BP reading involves identifying a threshold indicative of an accuracy of the sensed PPG readings, and computing an offset and gain of the stream of sensed PPG readings. Periodically or upon each reading, the system 162 compares the offset and gain to the threshold, and determines the inaccuracy based on the comparison of at least one of the offset and gain, and thus the need to commence the refresh reading 160.

FIG. 3 shows a flowchart of model 130 training as in FIG. 2A in the form of a schematic diagram of the flow steps consisting of feature extraction, training, hyperparameter selection, and testing. Referring to FIG. 3, at step 302, data including PPG and ECG readings is gathered. Training and test data segments are selected at step 304. The 7 randomly selected test segments in the test dataset were placed in sequential order, 1, 2, 3, . . . , 7. Not all segments reserved for testing were used as such, and segment 1 from each subject was designated as the personalization segment that was never used for testing. Personalization consisted of the ABP mean and SD features, whose values were always taken from a data segment occurring sooner in time than the test segment, from the same subject. By default, the mean and SD of the ABP values for test segment 1 (the personalization segment) were set aside. Then, test segment 2 was paired with 5, 3 with 6, and 4 with 7. If the mean PPG value from segment 2 differed by ≤5% from the mean PPG value of segment 1, then the default mean and standard deviation ABP from segment 1 was the personalization input for segment 2, and segment 5 was unused for testing. Otherwise, the ABP mean and ABP standard deviation from segment 2 were the personalization inputs for segment 5 (making segment 2 the personalization trial), and segment 2 was unused for testing. (We refer to this switch in the personalization trial as re-calibration.) The other 32 input features always came from the test segment used. Similar conditional testing was performed on the two other paired segments, with segment 1 always being used as the default. Three test results were thus computed per subject. This personalization ensured that subject-specific information was inserted as an input layer to the algorithm. The ABP mean and SD values from a test segment were never used when testing that segment. For calibration-free models, testing using the 32 input features was conducted on the same 3 segments per subject, for which testing was performed using the personalized method. Doing so balanced the test dataset between the personalized and calibration-free methods.

At step 306, the features for training are identified, including the Catch 22 features, as well as the mean, standard deviation and other personalization features. Training the model 130′ occurs at step 308, and hyperparameters determined at step 310. Prediction of BP 111 (SBP and DBP) occurs at step 312.

Note that a cutoff of a 5% change in the mean PPG signal in personalization may be selected empirically by evaluating the estimated systolic BP error SD using the ResNET algorithm as the mean PPG threshold was varied. The systolic error SD for change in mean PPG by 4%, 5%, and 6% was found to be 6.56 mm Hg, 7.91 mm Hg, and 8.34 mm Hg, respectively. Thus, a 5% change in mean PPG was used as it gave a systolic error SD<8 mm Hg as required by the AAMI (Association for the Advancement of Medical Instrumentation) criteria.

For each combination of the ML algorithm and model training approach (personalized, calibration-free), measured data includes test data bias (mean error or ME), mean absolute error (MAE), and SD error between estimated SBP and, separately, DBP in comparison with the corresponding labelled ABP data.

$$ME ( Mean Error ) = \frac{1}{n} \cdot {\sum }_{i = 1}^{n} ( \hat{A} i - Ai ) \qquad ( 1 )$$

$$SD ( Standard Deviation ) = \sqrt{\frac{1}{n - 1} \cdot [ {\sum }_{i = 1}^{n} {( ( \hat{A} i - Ai ) - ME )}^{2} ]} \qquad ( 2 )$$

$$MAE ( Mean Absolute Error ) = \frac{1}{n} \cdot {\sum }_{i = 1}^{n} \vert ( \hat{A} i - Ai ) \vert \qquad ( 3 )$$

$$RE ( Residual Error ) = ( \hat{A} i - Ai ) \qquad ( 4 )$$

where n is the number of total estimations, Ai is the ith reference SBP or DBP value, and Âi is the ith SBP or DBP value estimated by the model.

ME and SD are estimators of the BP estimation bias, and the range of errors in which the model's error on the population resides, under the assumption of normally distributed residual errors. Because mean arterial pressure (MAP) is correlated with SBP and DBP, it need not be shown separately. We also computed the number of residual errors below 5 mm Hg, 10 mm Hg, and 15 mm Hg as required by the British Society of Hypertension standard for BP monitoring. Two-way analysis of variance with post hoc multiple comparison tests were used to test significant differences in the means (for each of the 3 ML algorithms), per subject per case (for each of the two calibration methods). Levene's test was similarly used to test for significant differences in the variances. In all tests, p<0.05 was considered as significant.

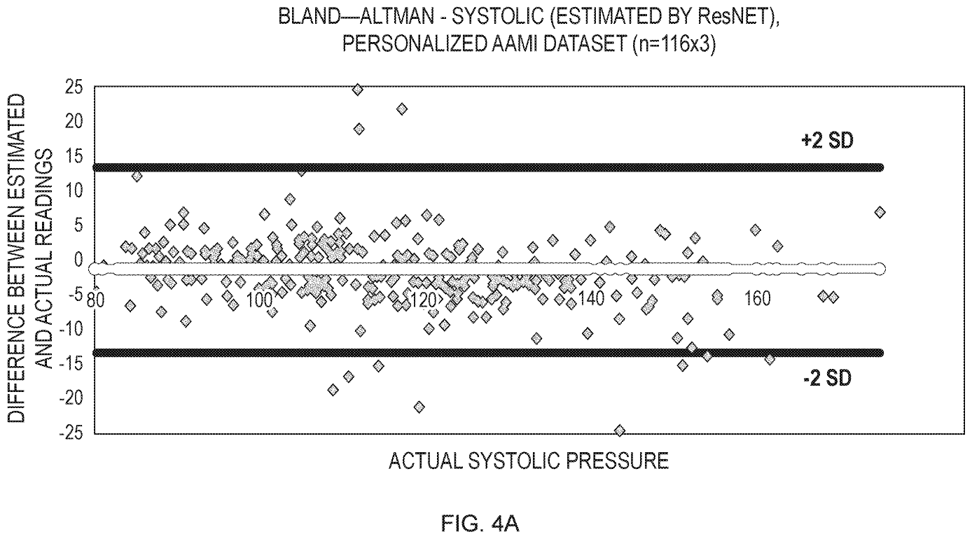

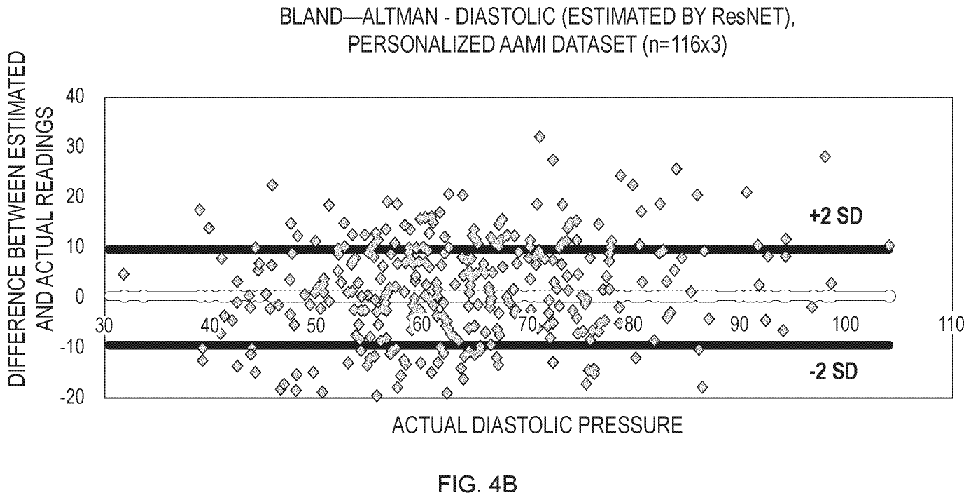

FIGS. 4A and 4B show results of computed BP compared to actual measurements using the model as in FIGS. 2A and 3. Referring to FIGS. 1-4B, FIG. 4A shows per-segment bias and standard deviation estimation test set errors, personalized method using ResNET, n=116×3. Blue lines show ±2 standard deviations, for an SBP comparison in FIG. 4A and DBP in FIG. 4B.

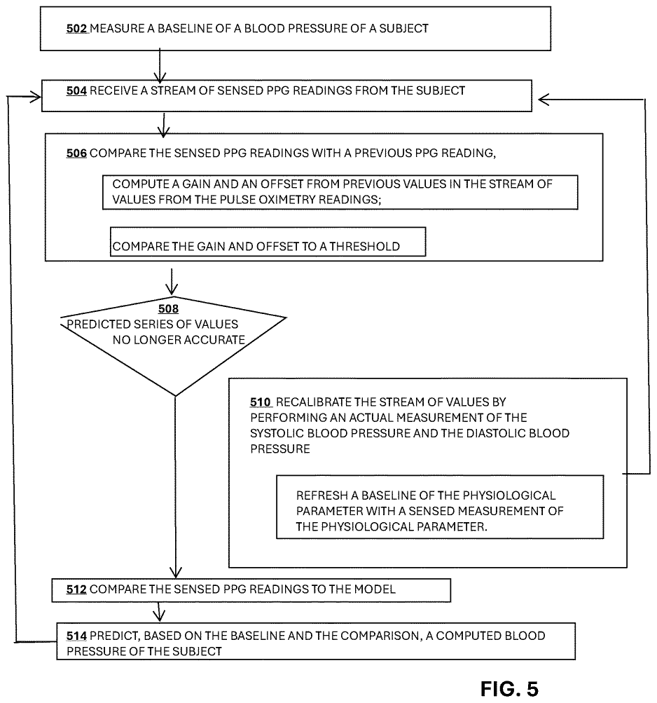

FIG. 5 shows a flowchart of model deployment as in FIG. 2B. FIG. 5 depicts a process flow on the system 162 or device for gathering PPG data, rendering PPG derived BP, and refreshing with a cuff reading when needed. Referring to FIGS. 1-5, the method of measuring blood pressure of a patient includes accumulating a database of physiological parameters related to blood pressure, the physiological parameters including photoplethysmographic (PPG) values, and generating the model 130′ for predicting blood pressure values based on the physiological parameters. At step 502, a baseline measurement of a blood pressure is gathered from a subject from a cuff or other actual reading. Once a baseline is established, a PPG sensor 140 is attached to a digit/finger for receiving a stream of sensed PPG readings from the subject, as depicted at step 504.

Upon receiving the PPG signals 142, the system 162 performs a comparison of the sensed PPG readings with a previous PPG reading for determining when the computed blood pressure becomes inaccurate, shown at step 506, and if so, commences a successive measurement for a refreshed baseline. This may include computing a gain and an offset from previous values in the stream of values from the pulse oximetry readings, comparing the gain and offset to a threshold; and determining that the predicted series of values is no longer accurate based on at least one of the gain or offset exceeding the threshold.

A check is performed, at step 508, to determine if the predicted series of values is no longer accurate, and if so, in response to determining an inaccuracy of the physiological parameter, recalibrating the stream of values by performing an actual measurement of the systolic blood pressure and the diastolic blood pressure, as depicted at step 510. Determining the inaccuracy may include comparing the mean and standard deviation to a threshold variance, and if the threshold variance of at least one of the mean and standard deviation is exceeded, recalibrating the stream of values by performing a true measurement of the physiological parameter. The system refreshes the baseline of the sensed parameter with a sensed measurement of the physiological parameter, and continues sensing at step 504 with the refreshed baseline.

At step 510, if the PPG reading has not drifted, the system 162 compares the sensed PPG readings 142 to the model 130′. The model 130′ predicts, based on the baseline and the comparison, the computed blood pressure 111 of the subject.

In alternative configurations, the stream of values 142 may be derived from ECG or other features. Also, internal BP measurements or skin mounted strain gauges may be employed for refresh/calibration. Ultrathin crystalline silicon-based omnidirectional strain gauges provide alternatives to photoplethysmography (PPG) for blood flow and volume monitoring include impedance plethysmography, which measures tissue electrical resistance changes, and specialized piezoelectric or semiconductor strain sensors (e.g., MEMS) that measure vascular expansion.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method of measuring physiological parameters, comprising:
receiving a stream of values indicative of a physiological parameter;
comparing the stream of values to a database of features related to the physiological parameter;
predicting, based on the comparison, a series of values of the physiological parameter; and
determining when the predicted series of values is no longer an accurate reflection of the physiological parameter.

2. The method of claim 1 further comprising, in response to determining an inaccuracy of the physiological parameter, recalibrating the stream of values by performing a true measurement of the physiological parameter.

3. The method of claim 2 wherein the stream of values includes a personalization value derived from previous actual measurements of the physiological parameter.

4. The method of claim 2 wherein the stream of values includes a baseline value derived from an actual measurement from a patient to which the physiological parameter applies; and
refreshing the baseline from a true measurement of the physiological parameter.

5. The method of claim 1 wherein the values in the stream of values are based on a measurement of dissolved blood gases.

6. The method of claim 1 wherein the stream of values includes pulse oximetry readings received from a subject, and the predicted series of values define a blood pressure of the subject.

7. The method of claim 6 wherein the physiological parameter further comprises a systolic blood pressure and a diastolic blood pressure, and the stream of values includes a baseline value derived from a previous systolic blood pressure and a previous diastolic blood pressure.

8. The method of claim 1 wherein the database of features includes a model of blood pressure parameters, the model of blood pressure parameters configured for predicting a systolic blood pressure and a diastolic blood pressure.

9. The method of claim 8 further comprising training the model on a dataset of features, the features including a mean of a photoplethysmography measurement of blood oxygen and a standard deviation of a photoplethysmography measurement of blood oxygen.

10. The method of claim 9 further comprising, in response to determining an inaccuracy of the physiological parameter, recalibrating the stream of values by performing an actual measurement of the systolic blood pressure and the diastolic blood pressure.

11. The method of claim 8 further comprising training the model on features including a mean of a photoplethysmography measurement of blood oxygen and a standard deviation of a photoplethysmography measurement of blood oxygen corresponding to at least one of a systolic blood pressure and the diastolic blood pressure.

12. The method of claim 1 wherein determining the inaccuracy includes comparing the mean and standard deviation to a threshold variance, and if the threshold variance of at least one of the mean and standard deviation is exceeded, recalibrating the stream of values by performing a true measurement of the physiological parameter.

13. The method of claim 6 further comprising:
computing a gain and an offset from previous values in the stream of values from the pulse oximetry readings;
comparing the gain and offset to a threshold;
determining that the predicted series of values is no longer accurate based on at least one of the gain or offset exceeding the threshold; and
refreshing a baseline of the physiological parameter with a sensed measurement of the physiological parameter.

14. A method of measuring blood pressure of a patient, comprising:
accumulating a database of physiological parameters related to blood pressure, the physiological parameters including photoplethysmographic (PPG) Photoplethysmography (PPG) values;
generating a model for predicting blood pressure values based on the physiological parameters;
measuring a baseline of a blood pressure of a subject;
receiving a stream of sensed PPG readings from the subject;
comparing the sensed PPG readings to the model
predicting, based on the baseline and the comparison, a computed blood pressure of the subject; and
determining, based on a comparison of the sensed PPG readings with a previous PPG reading, when the computed blood pressure becomes inaccurate, and if so, commencing a successive measurement for a refreshed baseline.

15. The method of claim 14 further comprising:
identifying a threshold indicative of an accuracy of the sensed PPG readings;
computing an offset and gain of the stream of sensed PPG readings;
comparing the offset and gain to the threshold; and
determining the inaccuracy based on the comparison of at least one of the offset and gain.

16. A cuffless blood pressure measurement and monitoring system, comprising:
an interface to a sensor configured for receiving a stream of values indicative of a physiological parameter;
a database of features related to the physiological parameter
a processor for comparing the stream of values to the database
a model having prediction logic configured to predict, based on the comparison, a series of values of the physiological parameter; and
comparison logic configured to determine when the predicted series of values is no longer an accurate reflection of the physiological parameter.
